(19) United States
(12) Patent Application Publication
(10) Pub. No.: US 2010/0298362 A1
(43) Pub. Date: Nov. 25, 2010

(54) PROCESS FOR THE PREPARATION OF 4-[6-(6-METHANESULFONYL-2-METHYL-PYRIDIN-3-YLAMINO)-5-METHOXY-PYRIMIDIN-4-YLOXY]-PIPERIDINE-1-CARBOXYLIC ACID ISOPROPYL ESTER

(76) Inventors: Xun Li, New Hope, PA (US); Ken Wells, Hillsborough, NJ (US); Shawn Branum, Easton, PA (US); Sandra Damon, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/783,927

(22) Filed: May 20, 2010

Related U.S. Application Data
(60) Provisional application No. 61/179,786, filed on May 20, 2009.

(51) Int. Cl. A61K 31/506; C07D 239/553; A61P 3/00

(52) U.S. Cl. 514/269; 544/319

(57) ABSTRACT

The present invention is directed to a process for the preparation of 4-[6-(6-methanesulfonyl-2-methyl-pyridin-3-ylamino)-5-methoxy-pyrimidin-4-yloxy]-piperidine-1-carboxylic acid isopropyl ester, useful as glucose dependent insulinotropic receptor agonist, for the treatment of metabolic-related disorders and complications thereof, such as, diabetes and obesity.

### CROSS REFERENCE TO RELATED APPLICATIONS

This application claims the benefit of U. S. Provisional Application 61/179,786, filed on May 20, 2009, which is incorporated by reference herein in its entirety.

### FIELD OF THE INVENTION

The present invention is directed to a process for the preparation of GPR119 agonist 4-[6-(6-methanesulfonyl-2-methyl-pyridin-3-ylamino)-5-methoxy-pyrimidin-4-yloxy]-piperidine-1-carboxylic acid isopropyl ester, useful as glucose dependent insulinotropic receptor agonist, for the treatment of metabolic-related disorders and complications thereof, such as, diabetes and obesity.

### BACKGROUND OF THE INVENTION

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are Type I (also referred to as insulin-dependent diabetes mellitus or IDDM) and Type II (also referred to as non-insulin-dependent diabetes mellitus or NIDDM). The etiology of the different types of diabetes is not the same; however, everyone with diabetes has two things in common: overproduction of glucose by the liver and little or no ability to move glucose out of the blood into the cells where it becomes the body's primary fuel. People who do not have diabetes rely on insulin, a hormone made in the pancreas, to move glucose from the blood into the cells of the body. However, people who have diabetes either don't produce insulin or can't efficiently use the insulin they produce; therefore, they can't move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

People with IDDM, which accounts for about 5% to 10% of those who have diabetes, don't produce insulin and therefore must inject insulin to keep their blood glucose levels normal. IDDM is characterized by low or undetectable levels of endogenous insulin production caused by destruction of the insulin-producing 13 cells of the pancreas, the characteristic that most readily distinguishes IDDM from NIDDM. IDDM, once termed juvenile-onset diabetes, strikes young and older adults alike.

Approximately 90 to 95% of people with diabetes have Type II (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (ROTWEIN, R. et al., “Polymorphism in the 5′ flanking region of the human insulin gene: a genetic marker for non-insulin dependent diabetes”, N. Engl. J. Med., 1983, pp 65-71, Vol. 308). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically unapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (RIMOIN, D. L., et. al., Emery and Rimoin's Principles and Practice of Medical Genetics 3^{rd} Ed., 1996, pp 1401-1402, Volume 1).

Many people with NIDDM have sedentary lifestyles and are obese; they weigh approximately 20% more than the recommended weight for their height and build. Furthermore, obesity is characterized by hyperinsulinemia and insulin resistance, a feature shared with NIDDM, hypertension and atherosclerosis.

Obesity and diabetes are among the most common human health problems in industrialized societies. In industrialized countries a third of the population is at least 20% overweight. In the United States, the percentage of obese people has increased from 25% at the end of the 1970s, to 33% at the beginning the 1990s. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increase insulin secretion balances insulin resistance and protects patients from hyperglycemia (LE STUNFF, C, et al., “Early Changes in Postprandial Insulin Secretion, Not in Insulin Sensitivity, Characterize Juvenile Obesity” Diabetes, 1994, pp 696-702, Vol. 43). However, after several decades, β cell function deteriorates and non-insulin-dependent diabetes develops in about 20% of the obese population (PEDERSON, P., “The Impact of Obesity on the Pathogenesis of Non-Insulin-Dependent Diabetes Mellitus: A Review of Current Hypotheses”, Diab. Metab. Rev., 1989, pp 505-509, Vol. 5) and (BRANCATI, F. L., et al., “Body Weight Patterns From 20 to 49 Years of Age and Subsequent Risk for Diabetes Mellitus: The Johns Hopkins Precursors Study”, Arch. Intern. Med., 1999, pp 957-963, Vol. 159). Given its high prevalence in modern societies, obesity has thus become the leading risk factor for NIDDM (HILL, J. O., et al., “Environmental contributions to the obesity epidemic”, Science, 1998, pp 1371-1374, Vol. 280). However, the factors which predispose a fraction of patients to alteration of insulin secretion in response to fat accumulation remain unknown.

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, obesity can also be defined on the basis of body fat content:

greater than 25% and 30% in males and females, respectively.

CLASSIFICATION OF WEIGHT BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (XENICAL™) and Sibutramine. Orlistat (a lipase inhibitor) inhibits fat absorption directly and tends to produce a high incidence of unpleasant (though relatively harmless) side-effects such as diarrhea. Sibutramine (a mixed 5-HT/noradrenaline reuptake inhibitor) can increase blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn after reports of preliminary evidence of heart valve abnormalities associated with their use. Accordingly, there is a need for the development of a safer anti-obesity agent.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of these conditions based on the prevention of obesity (PERRY, I. J., et al., “Prospective study of risk factors for development of non-insulin dependent diabetes in middle aged British men”, British Med J., 1995, pp 560-564, Vol. 310).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

Jones, R. M., et al., in PCT Publication WO2006/083491, published Oct. 8, 2006 disclose substituted pyridinyl and pyrimidinyl derivatives, compounds which bind to and modulate the activity of a GPCR and uses thereof.

### SUMMARY OF THE INVENTION

The present invention is directed to a process for the preparation of a compound of formula (I-S)

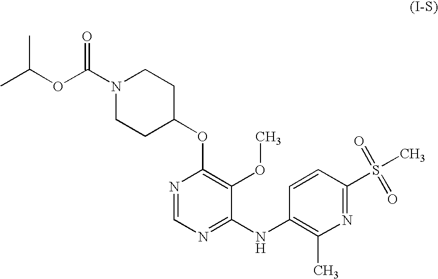

or a pharmaceutically acceptable salt, solvate or hydrate thereof; comprising

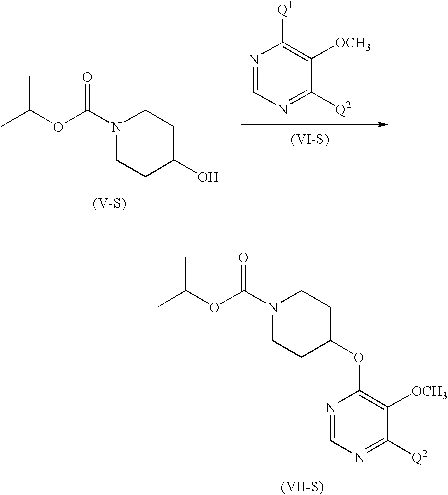

reacting a compound of formula (V-S) with a compound of formula (VI-S) wherein Q¹ and Q² are each an independently selected leaving group, in the presence of a first base, in a first organic solvent; to yield the corresponding compound of formula (VII-S);

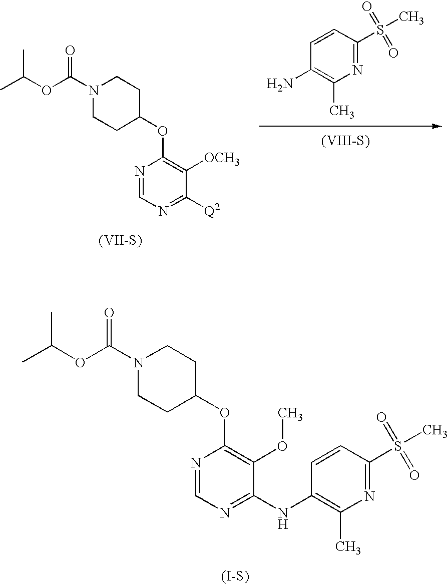

reacting the compound of formula (VII-S) with a compound of formula (VIII-S), in the presence of a second base; in the presence of a catalyst system; in a second organic solvent; to yield the corresponding compound of formula (I-S).

The present invention is further directed to a product prepared according to the process as described herein.

Illustrative of the invention is a pharmaceutical composition comprising a pharmaceutically acceptable carrier and a product prepared according to the process described herein. An illustration of the invention is a pharmaceutical composition made by mixing a product prepared according to the process described herein and a pharmaceutically acceptable carrier. Illustrating the invention is a process for making a pharmaceutical composition comprising mixing a product prepared according to the process described herein and a pharmaceutically acceptable carrier.

Exemplifying the invention are methods of treating a metabolic related disorder (selected from the group consisting of hyperlipidemia, type 1 diabetes, type 2 diabetes mellitus, idiopathic type 1 diabetes (Type 1 b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance) comprising administering to a subject in need thereof a therapeutically effective amount of a product prepared according to the process described herein or pharmaceutical composition as described herein.

The present invention is further directed to methods for decreasing food intake, inducing satiety, controlling weight gain or decreasing weight gain, comprising administering to a subject in need thereof a therapeutically effective amount of a product prepared according to the process described herein or pharmaceutical composition as described herein.

The present invention is further directed to the use of a product prepared according to the process as described herein in a method of treatment of the human or animal body by therapy.

The present invention is further directed to the use of a product prepared according to the process as described herein for use in a method of treatment of a metabolic related disorder.

The present invention is further directed to the use of a product prepared according to the process as described herein for use in a method of treatment of a metabolic related disorder selected from the group consisting of Type I diabetes, Type II diabetes, inadequate glucose tolerance, insulin resistance, hyperglycemia, hyperlipidemia, hypertriglyceridemia, hypercholesterfolemia, dyslipidemia and Syndrome X.

The present invention is further directed to the use of a product prepared according to the process as described herein for treatment of Type II diabetes.

The present invention is further directed to the use of a product prepared according to the process as described herein for use in a method of (a) decreasing food intake, (b) inducing satiety, (c) controlling weight gain, or (d) decreasing weight gain, in a subject in need thereof.

Another example of the present invention is the use of a product prepared according to the process described herein in the preparation of a medicament for treating a metabolic related disorders, in a subject in need thereof.

Another example of the present invention is the use of any of the compounds described herein in the preparation of a medicament for treating (a) type I diabetes, (b) type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, (j) syndrome X or (k), in a subject in need thereof.

The present invention is further directed to the use of a product prepared according to the process described herein for the preparation of a medicament for treating Type II diabetes, in a subject in need thereof.

The present invention is further directed to the use of a product prepared according to the process described herein for the preparation of a medicament for (a) decreasing food intake, (b) inducing satiety, (c) controlling weight gain, or (d) decreasing weight gain, in a subject in need thereof.

### DETAILED DESCRIPTION OF THE INVENTION

The present invention is directed to a process for the preparation of a compound of formula (I-S)

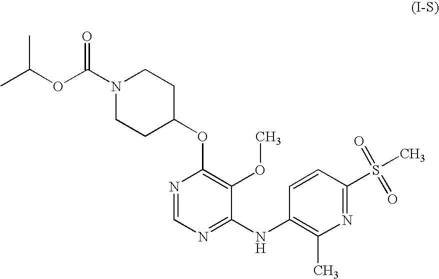

or pharmaceutically acceptable salt, solvate or hydrate thereof. The compound of formula (I-S), also known as 4-[6-(6-methanesulfonyl-2-methyl-pyridin-3-ylamino)-5-methoxy-pyrimidin-4-yloxy]-piperidine-1-carboxylic acid isopropyl ester, is a glucose dependent insulintropic receptor agonists useful in the treatment of metabolic related disorders.

Jones, R. M., et al., in US Patent Publication 2007/0167473 A1, published Jul. 19, 2007, which is incorporated by reference in its entirety herein, disclose the compound of formula (I-S), methods for the preparation of the compound of formula (I-S) and methods of treatment using the compound of formula (I-S). The compound of formula (I-S) is a selective GDIR (glucose-dependent insulin receptor) agonist useful for the treatment of glucose related disorders, including, but not limited to, Type I diabetes, Type II diabetes, inadequate glucose tolerance, insulin resistance, hyperglycemia, hyperlipidemia, hypertriglyceridemia, hypercholesterolemia, dyslipidemia, or Syndrome X.

The present invention is further directed to a compound prepared according to the process described herein. The present invention is further directed to methods for the treatment of metabolic related disorders comprising administering to a subject in need thereof, a therapeutically effective amount of a compound prepared according to the process described herein.

In some embodiments of the present invention, the metabolic-related disorder is selected from the group consisting of hyperlipidemia, type 1 diabetes, type 2 diabetes mellitus, idiopathic type 1 diabetes (Type 1 b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction, and impaired vascular compliance.

In another embodiment of the present invention, the metabolic related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia and Syndrome X. In an embodiment of the preset invention, the metabolic-related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X. In another embodiment of the present invention, the metabolic-related disorder is Type II diabetes. In another embodiment of the present invention, the metabolic-related disorder is hyperglycemia. In another embodiment of the present invention, the metabolic-related disorder is hyperlipidemia. In another embodiment of the present invention, the metabolic-related disorder is hypertriglyceridemia. In another embodiment of the present invention, the metabolic-related disorder is Type I diabetes. In another embodiment of the present invention, the metabolic-related disorder is dyslipidemia. In another embodiment of the present invention, the metabolic-related disorder is Syndrome X.

The present invention is further directed to methods for decreasing food intake, inducing satiety, controlling weight gain or decreasing weight gain, comprising administering to a subject in need thereof a therapeutically effective amount of a product prepared according to the process described herein or pharmaceutical composition as described herein. In another embodiment, the present invention is directed to the use of a compound prepared according to the process described herein for the treatment of a metabolic-related disorder, wherein the metabolic related disorder is obesity.

In some embodiments, the present invention is directed to method of treating human patients whose body mass index is in the range of from about 18.5 to about 45. In some embodiments, the human has a body mass index of from about 25 to about 45. In some embodiments, the human has a body mass index of from about 30 to about 45. In some embodiments, the human has a body mass index of from about 35 to about 45.

In addition to the foregoing beneficial uses, a product prepared according to the process described herein is further useful in the treatment of additional diseases, including, without limitation, the following.

The most significant pathologies in Type II diabetes are impaired insulin signaling at its target tissues (“insulin resistance”) and failure of the insulin-producing cells of the pancreas to secrete an appropriate degree of insulin in response to a hyperglycemia signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP1, GIP and PACAP. These hormones act via their cognate G-protein coupled receptor to stimulate the production of cAMP in pancreatic β-cells. The increased cAMP does not appear to result in stimulation of insulin release during the fasting or preprandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes.

It is also established that increased cAMP, for example as a result of GLP1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the co-development of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

One aspect of the present invention pertains to methods of modulating a GPR119 receptor in an individual comprising contacting the receptor with a product prepared according to the process described herein. In some embodiments, the modulation of the GPR119 receptor is treatment of a metabolic-related disorder and complications thereof. In some embodiments, the metabolic-related disorder is type I diabetes, type II diabetes, inadequate glucose tolerance, insulin resistance, hyperglycemia, hyperlipidemia, hypertriglyceridemia, hypercholesterolemia, dyslipidemia or syndrome X. In some embodiments, the metabolic-related disorder is type II diabetes. In some embodiments, the metabolic-related disorder is hyperglycemia. In some embodiments, the metabolic-related disorder is hyperlipidemia. In some embodiments, the metabolic-related disorder is hypertriglyceridemia. In some embodiments, the metabolic-related disorder is type I diabetes. In some embodiments, the metabolic-related disorder is dyslipidemia. In some embodiments, the metabolic-related disorder is syndrome X. In some embodiments, the individual is a mammal. In some embodiments, the mammal is a human.

In another embodiment, the present invention is directed to the use of a compound prepared according to the process described herein for reducing food intake, inducing satiety, controlling weight gain, reducing weight gain.

Some embodiments of the present invention include a method of modulating a GPR119 receptor in an individual comprising contacting the receptor with a product prepared according to the process described herein, wherein the modulation of the GPR119 receptor reduces food intake of the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

Some embodiments of the present invention include a method of modulating a GPR119 receptor in an individual comprising contacting the receptor with a product prepared according to the process described herein, wherein the modulation of the GPR119 receptor induces satiety in the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

Some embodiments of the present invention include a method of modulating a GPR119 receptor in an individual comprising contacting the receptor with a product prepared according to the process described herein, wherein the modulation of the GPR119 receptor controls or reduces weight gain of the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The compound according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed in BERGE, S. M., et al., “Pharmaceutical Salts”, J. of Pharm. Sci., 1977, pp 66-18, Vol. 2, incorporated herein by reference in its entirety.

The acid addition salts may be obtained as the direct product of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

In addition, compounds according to the invention may optionally exist as pharmaceutically acceptable basic addition salts. For example, these salts can be prepared in situ during the final isolation and purification of the compounds of the invention, or separately by reacting an acidic moiety, such as a carboxylic acid, with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

The compound of the present invention can further be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

The compound of the present invention additionally includes tautomeric forms, such as keto-enol tautomers, and the like. Tautomeric forms can be in equilibrium or sterically locked into one form by appropriate substitution. It is understood that the various tautomeric forms are within the scope of the compounds of the present invention. The compound of the present invention also include all isotopes of atoms occurring in the intermediates and/or final compounds. Isotopes include those atoms having the same atomic number but different mass numbers. For example, isotopes of hydrogen include deuterium and tritium.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

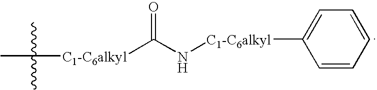

Abbreviations used in the specification, particularly the Schemes and Examples, are as follows:

BMI=Body Mass Index

DMA=Dimethylacetamide

DMF=N,N-Dimethylformamide

DMSO=Dimethylsulfoxide

EOD=Early-Onset Type 2 Diabetes

Et₂O=Diethyl ether

EtOAc=Ethyl acetate

IDDM=Insulin Dependent Diabetes Mellitus

IGT=Impaired Glucose Tolerance

KO-t-Bu=Potassium tert-Butoxide

LADA=Latent Autoimmune Diabetes in Adults

LDA=Lithium Diisopropylamide

LiHMDS=Lithium Hexamethyldisilazane

NaO-t-Bu=Sodium tert-Butoxide

NIDDM=Non-Insulin Dependent Diabetes Mellitus

NMP=N-methyl-2-pyrrolidinone

Pd₂(dba)₃=Tris(dibenzylideneacetone)dipalladium(0)

Pd(OAc)₂=Palladium acetate

THF=Tetrahydrofuran

YOAD=Youth-Onset Atypical Diabetes

As used herein, unless otherwise noted, the term “isolated form” shall mean that the compound is present in a form which is separate from any solid mixture with another compound(s), solvent system or biological environment. In an embodiment of the present invention, the compound of formula (I-S) is prepared as an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I-S) is prepared as a substantially pure form.

As used herein, unless otherwise noted, the term “substantially free of a corresponding salt form(s)” when used to described the compound of formula (I) shall mean that mole percent of the corresponding salt form(s) in the isolated compound of formula (I) is less than about 5 mole percent, preferably less than about 2 mole percent, more preferably, less than about 0.5 mole percent, most preferably less than about 0.1 mole percent. In an embodiment of the present invention, the compound of formula (I-S) is present in a form which is substantially free of corresponding salt forms.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follows herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, CI, I, F, mesylate, tosylate, triflate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

For example, terminal amino or alkylamino groups may be protected with a suitably selected nitrogen protecting group. As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

In another example, terminal hydroxy groups may be protected with a suitably selected oxygen protecting group. As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α-max])×100.

The present invention is directed to a process for the preparation of a compound of formula (I-S) as outlined in more detail in Scheme 1, below.

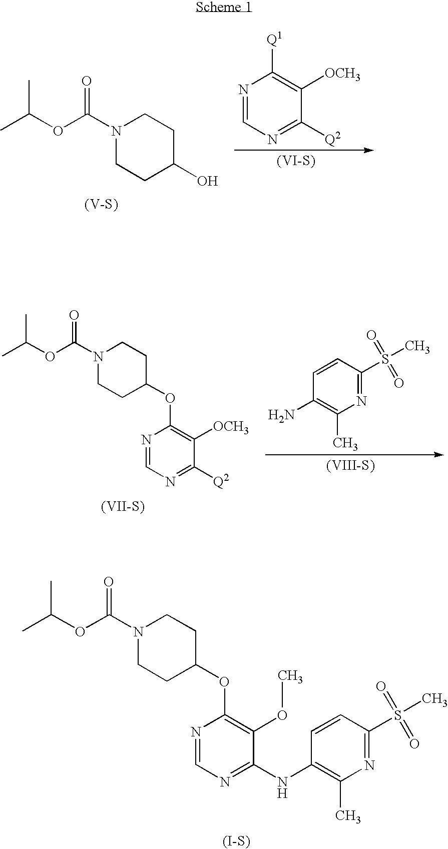

Accordingly, a suitably substituted compound of formula (V-S), a known compound or compound prepared by known methods, is reacted with a suitably substituted compound of formula (VI-S), wherein Q¹ and Q² are each an independently selected leaving group such as chloro, fluoro, bromo, iodo, methanesulfonate (mesylate), p-toluenesulfonate (tosylate), trifluoromethyanesulfonate (triflate) and the like, more preferably Q¹ and Q² are the same and are each chloro; a known compound or compound prepared by known methods; wherein the compound of formula (VI-S) is preferably present in an amount in the range of from about 0.5 to about 3.0 molar equivalents, more preferably in an amount in the range of from about 0.75 to about 1.5 molar equivalents, more preferably still, in an amount of about 1.0 molar equivalents;

in the presence of a suitably selected first base such as NaH, NaOH, KO-t-Bu, LDA, LIHMDS, Na-t-amylate, and the like, preferably NaH or KO-t-Bu, more preferably KO-t-Bu; wherein the first base is preferably present in an amount in the range of from about 0.5 to about 2.0 molar equivalents, more preferably in an amount in the range of from about 1.0 to about 1.2 molar equivalents, more preferably still, in an amount of about 1.1 molar equivalents;

in a first organic solvent such as DMF, THF, toluene, NMP, DMA, heptane, and the like, preferably toluene or DMF, more preferably toluene; preferably at a temperature in the range of from about −25° C. to about room temperature, more preferably at about 0° C. to about room temperature, more preferably at a temperature of about 10° C.; to yield the corresponding compound of formula (VII-S).

The compound of formula (VII-S) is reacted with a suitably substituted compound of formula (VIII-S), a known compound or compound prepared by known methods; wherein the compound of formula (VIII-S) is preferably present in an amount in the range of from about 0.5 to about 2.0 molar equivalents, more preferably in an amount in the range of from about 0.75 to about 1.25 molar equivalents, more preferably about 1.0 molar equivalents;

in the presence of a suitably selected second base, preferably an inorganic base such as NaO-t-Bu, KO-t-Bu, NaH, KH, Na-t-amylate, NaHCO₃ and the like, more preferably NaO-t-Bu; wherein the second base is preferably present in an amount in the range of from about 1.0 to about 3.0 molar equivalents, more preferably in an amount in the range of from about 1.1 to about 2.0 molar equivalents, more preferably in an amount of about 1.5 molar equivalents;

in the presence of a catalyst system comprising a suitably selected metal catalyst such as palladium, copper, nickel, iron, tungsten, manganese, and the like; preferably in the presence of a catalyst system comprising palladium and a phosphine ligand, such as a mixture of palladium acetate and bis(2-diphenylphosphinophenyl)ether acetate, palladium chloride, Pd₂(dba)₃, and the like, more preferably, a mixture of palladium acetate and bis(2-diphenylphosphinophenyl)ether, most preferably a mixture of 4 mol% bis(2-diphenylphosphinophenyl)ether and 2 mol % palladium acetate;

in a second organic solvent such as toluene, 1,4-dioxane, THF, and the like, preferably toluene; preferably at a temperature in the range of from about room temperature to about solvent reflux temperature, more preferably at a temperature in the range of from about 40° C. to about 90° C., more preferably still, at a temperature of about 60° C.; to yield the corresponding compound of formula (I-S).

Preferably, the compound of formula (I-S) is isolated according to known methods, for example by filtration or column chromatography. Preferably, the compound of formula (I-S) is further crystallized according to known methods, from a suitably selected organic solvent or mixture of organic solvents. Preferably, the compound of formula (I-S) is further purified according to known methods, for example by recrystallization from a suitably selected organic solvent or mixture of organic solvents.

The present invention further comprises pharmaceutical compositions containing a compound prepared according to the process described herein with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 to about 1000 mg or any amount of range therein, and may be given at a dosage of from about 0.01 to about 500 mg/kg/day, or any amount or range therein, preferably from about 0.5 to about 300 mg/kg/day, or any amount or range therein, more preferably from about 1.0 to about 100 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 to about 1000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating metabolic related disorders described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably about 1.0 to about 500 mg of the compound, or anyamount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I-S) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of metabolic related disorders is required.

The daily dosage of the products may be varied over a wide range from about 0.01 to about 5,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1,000 milligrams of the active ingredient for the symptomatic adjustment of the dosage to the patient to be treated. An effective amount of the drug is ordinarily supplied at a dosage level of from about 0.01 mg/kg to about 500 mg/kg of body weight per day, or any range amount or therein. Preferably, the range is from about 0.01 to about 300.0 mg/kg of body weight per day, or any amount or range therein. More preferably, from about 0.5 to about 300.0 mg/kg of body weight per day, or any amount or range therein. More preferably, from about 1.0 to about 100.0 mg/kg of body weight per day, or any amount or range therein. More preferably, from about 10.0 to about 50.0 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trails

including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 4-(6-Chloro-5-methoxy-pyrimidin-4-yloxy)-piperidine-1-carboxylic acid isopropyl ester

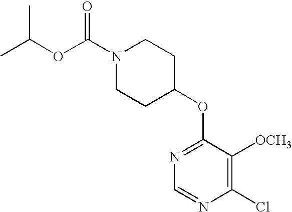

A 500 ml, 4-necked flask equipped with thermometer, mechanical stirrer and condenser with gas inlet was purged with N₂ and charged with NaH (4.4 g; 0.11 mol) and N,N-dimethylformamide (50 ml). In a separate flask were dissolved 4-hydroxy-piperidine-1-carboxylic acid isopropyl ester (18.7 g; 0.1 mol) and 4,6-dichloro-5-methoxy-pyrimidine (17.9 g; 0.1 mol) in DMF (50 ml; 0.5 L/mol). The prepared solution was then added dropwise to the above-mentioned NaH/DMF suspension while maintaining the temperature between −10 and −5° C. The resulting mixture is then stirred for one hour, then allowed to warm up to room temperature and stirred for 17 hours. Water (300 ml; 3 L/mol) was added dropwise while maintaining the temperature between 15-30° C. by cooling with tap water. Heptane (125 ml; 1.25 L/mol) was added and the resulting mixture was heated up to 55° C. The aqueous layer was discarded; the organic layer was cooled down to 20° C. and stirred for another 3-20 h. The resulting precipitate was filtered and dried in vacuum at 50° C. for 20 h to yield the title compound.

### Example 2

### 4-(6-Chloro-5-methoxy-pyrimidin-4-yloxy)-piperidine-1-carboxylic acid isopropyl ester

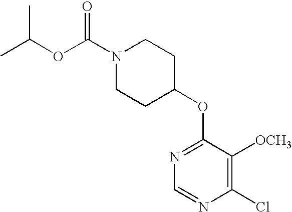

A 1 L, 4-necked flask equipped with thermometer, mechanical stirrer and condenser with gas inlet was purged with N₂ and charged with 4-hydroxy-piperidine-1-carboxylic acid isopropyl ester (18.7 g; 0.1 mol), toluene (200 ml; 2 L/mol) and 4,6-dichloro-5-methoxy-pyrimidine (17.8 g; 1 eq.). The resulting mixture was cooled down to 10° C. A solution of 1N KO-t-Bu in THF (110 ml; 1.1 eq.) was then added dropwise over 10 min. After 30 min, the resulting mixture was allowed to heat to room temperature. After 1 h at room temperature, the resulting mixture was quenched with water (200 ml; 2 L/mol) and stirred for 10 min. The aqueous layer was discarded and the organic layer was evaporated to dryness under vacuum. Heptane (60 ml; 0.6 L/mol) was added and the resulting mixture was heated up to 55-60° C. (colorless transparent solution), then cooled to 45° C., seeded with polymorph form IV and further cooled down to room temperature over 2 h. The resulting crystallized product (heavy crystals) was filtered off and washed with heptane (15 ml; 0.15 L/mol), then dried (45° C., vacuum, 2 hours) to yield the title compound.

### Example 3

### 4-[6-(6-Methanesulfonyl-2-methyl-pyridin-3-ylamino)-5-methoxy-pyrimidin-4-yloxy]-piperidine-1-carboxylic acid isopropyl ester

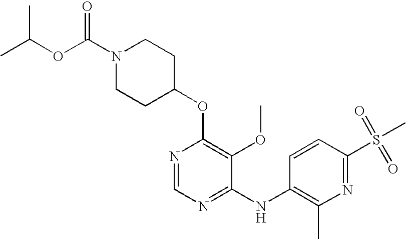

### STEP A: Catalyst Preparation

A 250 ml, 4-necked flask equipped with thermometer, mechanical stirrer and condenser with gas inlet is purged with argon and charged with toluene (120 ml; 1 L/mol). The toluene was degassed by bubbling with argon for 15 min. Bis(2-diphenylphosphinophenyl) ether (2.59 g; 4 mol %) was dissolved under a stream of argon. Palladium(II) acetate (0.54 g; 2 mol %) was added in one portion; with the orange powder observed to go into solution. After a few minutes a solid appeared and a yellow suspension was observed.

### STEP B: Coupling

A 250 ml, 4-necked flask equipped with thermometer, mechanical stirrer and condenser with gas inlet was purged with Argon and then charged with toluene (360 ml, 3 L/mol). The toluene was degassed by bubbling with argon for 15 minutes. 4-(6-Chloro-5-methoxy-pyrimidin-4-yloxy)-piperidine-1-carboxylic acid isopropyl ester (39.6 g; 0.12 mol), 6-methanesulfonyl-2-methyl-pyridin-3-ylamine (22.3 g; 0.12 mol) and NaO-t-Bu (17.3 g; 1.5 eq.) were added at room temperature. The resulting suspension was heated to 60° C. while being degassed by bubbling with Argon. The catalyst suspension prepared as in STEP A above was added in one portion to the reaction mixture (the catalyst suspension is fluid enough to be added via an addition funnel). After 30 minutes, the Ar-stream was stopped and switched to a N₂ stream. The resulting mixture was stirred over 16-18 h at 60° C.

Water (120 ml; 1 L/mol) was then added to the hot reaction mixture while maintaining the temperature at 60° C. The solid was observed to dissolve. A brown organic layer was obtained after discarding the aqueous one together with the black interface. Aqueous 1M HCl (120 ml; 1.2 eq.) was added and the color of the mixture was observed to go from brown to light brown. The resulting mixture was then heated to 60° C. The same extraction procedure was carried out a second time (aqueous layer discarded, organic layer heated to 60° C.).

To the resulting mixture was added water (120 ml, 1.2 L/mol) and the aqueous layer was discarded. To the organic layer was then added sodium sulfate (9.0 g; 75 g/mol), Silica Gel Thiol 3 (11.4 g; 95 g/mol) and NORIT® A SUPRA (2.4 g; 20 g/mol) and the resulting mixture stirred for 30 min at 60° C.; then filtered over dicalite. The filter cake was rinsed with toluene (12 ml; 0.1 L/mol) to yield the title compound as a solid.

### Example 4

### Crystallization of 4-[6-(6-Methanesulfonyl-2-methyl-pyridin-3-ylamino)-5-methoxy-pyrimidin-4-yloxy]-piperidine-1-carboxylic acid isopropyl ester

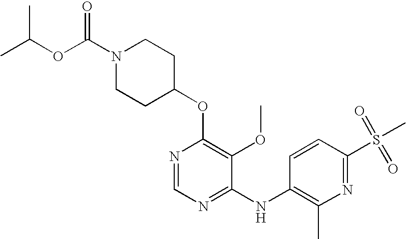

Toluene was stripped off (+/−15% of total toluene) from the filtercake prepared as in Example 3 above, and then isopropanol (640 ml; 5.33 L/mol) was added. To the resulting solution was then added Silica Gel Thiol 3 (11.4 g; 95 g/mol) and NORIT® A SUPRA (2.4 g; 20 g/mol) and the resulting mixture stirred for 30 min at 60° C., then filtered over dicalite. The filtercake was rinsed with isopropanol (12 ml; 0.1 L/mol). The remaining toluene was distilled with the isopropanol (azeotrope at 81° C.). The residual solution was left to cool down slowly to room temperature. The resulting mixture was filtered on a filter paper and the solid rinsed with isopropanol (24 ml; 0.2 L/mol). The solid was dried for 18 h in an oven under vacuum at 50° C. with a stream of N₂ to yield the title compound as a crystalline solid.

### Example 5

### Recrystallization of 4-[6-(6-Methanesulfonyl-2-methyl-pyridin-3-ylamino)-5-methoxy-pyrimidin-4-yloxy]-piperidine-1-carboxylic acid isopropyl ester

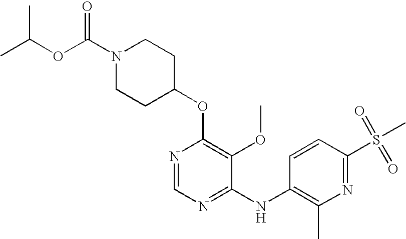

A 250 ml, 4-necked flask equipped with thermometer, mechanical stirrer and condenser with a gas inlet was purged with N₂ and charged with ethanol (80 ml; 4.8 L/mol), 4-[6-(6-methanesulfonyl-2-methyl-pyridin-3-ylamino)-5-methoxy-pyrimidin-4-ylmethyl]piperidine-1-carboxylic acid isopropyl ester (8.0 g; 0.0167 mol) and Silica Gel Thiol 3 (2.5 g; 150 g/mol). The resulting suspension was heated to reflux (75-76° C.), stirred at this temperature for 45 min, then cooled to 70-72° C. The resulting warm suspension was filtered over dicalite to remove the silica gel and the flask was rinsed with ethanol (20 ml; 1.2 L/mol).

The filtrate was then reheated to reflux and stirred for 15 min; then to 57° C. over 1 h. The resulting solution was seeded, stirred at 50-55° C. for 30 minutes, cooled down further to 20° C. over 2 h then further cooled to 5° C. over 1 h. The resulting mixture was stirred at 5° C. for 1 h, filtered and dried (40° C., vacuum, N₂, 18 h) to yield the title compound as a crystalline solid.

### Example 6

### 4-(6-Chloro-5-methoxy-pyrimidin-4-yloxy)-piperidine-1-carboxylic acid isopropyl ester

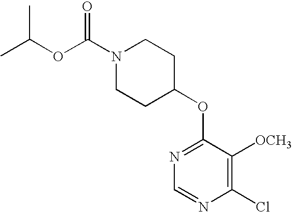

A 5-L 4-neck flask equipped with a thermocouple controller, an overhead mechanical stirrer, and a nitrogen inlet/outlet was charged with isopropyl 4-hydroxypiperidine-1-carboxylate (95.5 g, 0.510 mol) and 4,6 dichloro-5-methoxypyrimidine (96 g, 0.536 mol) in THF (1.4 L). The resulting homogenous mixture was cooled to 5° C. and slowly treated with 1M KOt-Bu (511 mL, 0.511 mol) over a 1 hour period, while maintaining the temperature <10° C. The resulting mixture was stirred for 1 hour at <10° C. The reaction was then quenched with NH₄Cl (1 L), diluted with Et₂O (1.5 L), and then by H₂O (500 mL). The organic layer was washed with brine (1 L), dried over MgSO₄, filtered and evaporated to dryness to yield the title compound as a residue.

The residue was triturated with heptane (500 mL) and the resulting solid was isolated by filtration. The cake was washed with heptane (3×200 mL). The solid was placed in vacuum oven for 18 hours at 40° C. to yield 4-(6-chloro-5-methoxy-pyrimidin-4-yloxy)-piperidine-1-carboxylic acid isopropyl ester as a yellowish solid. MS: 330.8 MW+H⁺

¹H NMR (400 MHz, CDCL₃) δ 8.27 (s, 1H), 5.39 (m, 1H), 4.94 (oct, J=6.2 Hz, 1H), 3.91 (s, 3H), 3.79 (m, 2H), 3.40 (m, 2H), 2.02 (m, 2H), 1.83 (m, 2H), 1.26 (d, J=6.2 Hz, 6H).

### Example 7

### 6-Methanesulfonyl-2-methyl-pyridin-3-ylamine

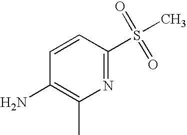

A 5-L 4-neck flask equipped with a thermocouple controller, an overhead mechanical stirrer, a condenser, and a nitrogen inlet/outlet was charged with sodium methanesulfinate (568 g, 4.73 mol), copper(1) trifluoromethanesulfonate benzene complex (70 g, 0.139 mol), N,N′-dimethylethylenediamine (12.3 g, 0.139 mol), and bromo-amine (260 g, 1.39 mol) in DMSO (800 mL). The resulting mixture was heated to 150° C. for 1 hour. The resulting mixture was then diluted with H₂O (1.5 L) and extracted with EtOAc (6×2 L). The combined organic layers were evaporated to dryness to yield a residue. The residue was purified via ISCO Prep chromatography system. The product containing fractions were combined and evaporated to dryness. The resulting product (residue) was placed in vacuum oven at 40° C. for 18 hours to yield 6-methanesulfonyl-2-methyl-pyridin-3-ylamine as a as a brownish solid. MS: 187.1 MW+H⁺

### Example 8

### 4-[6-(6-Methanesulfonyl-2-methyl-pyridin-3-ylamino)-5-methoxy-pyrimidin-4-yloxy]-piperidine-1-carboxylic acid isopropyl ester

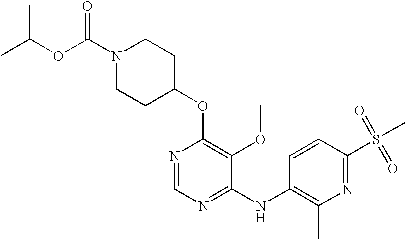

STEP A: Catalyst preparation

A 5-L 3-neck flask equipped with an overhead mechanical stirrer, N₂ inlet/outlet adapter, reflux condenser, and thermocouple was charged with 1,4-dioxane (715 mL) and degassed for 10 min. 1,1′-Bis (di-tert-butylphosphino) ferrocene (22.9 g, 0.046 mol), and Pd(OAc)₂ (5.1 g, 0.023 mol) were added at room temperature and the resulting mixture was degassed and purged with N₂ three times. The resulting heterogeneous solution was heated to 75° C. and stirred for 30 min. The resulting mixture was cooled to room temperature and sodium t-butoxide (62.5 g, 0.65 mol) was added all at once. The catalyst solution was degassed and purged with N₂ three times.

### STEP B: Coupling

4-(6-Chloro-5-methoxy-pyrimidin-4-yloxy)-piperidine-1-carboxylic acid isopropyl ester (143.0 g, 0.43 mol) and 6-methanesulfonyl-2-methyl-pyridin-3-ylamine (80.8 g, 0.43 mol) were added together with 1,4-dioxane (715 mL) to the catalyst solution prepared in STEP A above, via addition funnel over 10 min. The resulting mixture was heated to 50° C. for 18 hours. The resulting mixture was then cooled to room temperature, poured into EtOAc (2 L), and washed with 1N HCl (2×500 mL). The organic layer was then washed with brine (500 mL), dried over MgSO₄, filtered, and concentrated to dryness to yield a residue. The residue was purified using the Isco LC prep (2×1.5 kg SiO₂, 5 column volumes 50:50 EtOAc, 2 columns volumes 60:40 EtOAc:heptane, 20 column volumes 67:33 EtOAc:heptane, 254 nm wavelength, and 250 mL/min flow rate.) to yield 4-[6-(6-methanesulfonyl-2-methyl-pyridin-3-ylamino)-5-methoxy-pyrimidin-4-ylmethyl]piperidine-1-carboxylic acid isopropyl ester as a yellow solid. MS 478.2 MW+H⁺

### STEP C: Recrystallization

The chromatographed 4-[6-(6-methanesulfonyl-2-methyl-pyridin-3-ylamino)-5-methoxy-pyrimidin-4-ylmethyl]piperidine-1-carboxylic acid isopropyl ester was recrystallized in EtOAc (350 mL) and heptane (450 mL). The slurry was heated to 75° C. for ½ hr. The slurry was then cooled to room temperature over 30 min and aged 30 min. The resulting thick slurry was filtered, rinsed with heptane (3×25 mL), and put into a vacuum oven at 40° C. for 36 hours to yield 4-[6-(6-methanesulfonyl-2-methyl-pyridin-3-ylamino)-5-methoxy-pyrimidin-4-ylmethyl]piperidine-1-carboxylic acid isopropyl ester as a solid.

¹ H NMR (400 MHz, DMSO-d₆) δ 8.67 (s, 1H), 8.25 (d, J=8.3 Hz, 1H), 8.07 (s, 1H), 7.90 (d, J=8.3 Hz, 1H), 5.31 (m, 1H), 4.80 (sep, J=6.2 Hz, 1H), 3.87 (s, 3H), 3.68 (m, 2H), 3.33 (m, 2H),3.26 (s, 3H),2.54 (s, 3H), 1.98 (m, 2H), 1.68 (m, 2H),1.21 (d, J=6.2 Hz, 6H).

¹³CNMR (d₆-DMSO, 100 MHz) 159.3, 154.3, 154.2, 153.4, 151.5, 150.9, 137.7, 132.7, 125.2, 119.0, 71.0, 67.9, 60.0, 40.6, 40.1, 30.3, 22.0, 21.1.

Chemical Analysis for C₂₁ F₂₉N₅O₆S:
Calculated: C, 52.60: H, 6.10; N, 14.60; S, 6.68;
Measured: C, 52.61; H, 6.11; N, 14.

### Example 9

### 4-(6-Chloro-5-methoxy-pyrimidin-4-yloxy)-piperidine-1-carboxylic acid isopropyl ester

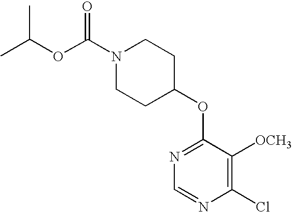

A 5-L 4-neck round bottom flask equipped with a thermocouple, a mechanical stirrer, a pressure-equalized dropping funnel, and a nitrogen inlet/outlet adapter was charged with NaH (34.3 g, 0.86 mol) and DMF (1.0 L). After cooling to 0° C., a solution of isopropyl 4-hydroxypiperidine-1-carboxylate (162.0 g, 0.86 mol) in DMF (0.4 L) was added dropwise via an additional dropping funnel over 1.5 hours. After stirring for an additional 1 hour at 0° C. the resulting mixture was cooled to −30° C. 4,6 Dichloro-5-methoxypyrimidine (154.9 g, 0.84 mol) was added to the resulting mixture in one portion. After the addition, the mixture was warmed to 20° C. over 2 hours and then stirred at 20° C. for 3 hours. The resulting mixture was then transferred to a 12-L flask. Water (7 L) was added, with stirring, resulting in the formation of a solid precipitate. The precipitate was collected by filtration and air-dried for 48 hours to yield the title compound as a light yellow solid.

The light yellow solid was crystallized in heptane (1.25 L) at 60° C. and then cooled to 15° C. The resulting white crystals were collected by filtration and then air dried for 24 hours, to yield the title compound. MS: 330.11 M+H⁺

### Example 10

### Oral Formulation—Prophetic Example

As a specific embodiment of an oral composition, 100 mg of the compound prepared as in Example 3 or 4 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A process for the preparation of a compound of formula (I-S) 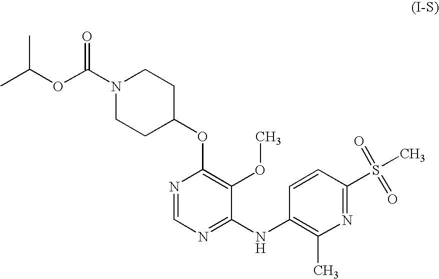
or a pharmaceutically acceptable salt, solvate or hydrate thereof; comprising 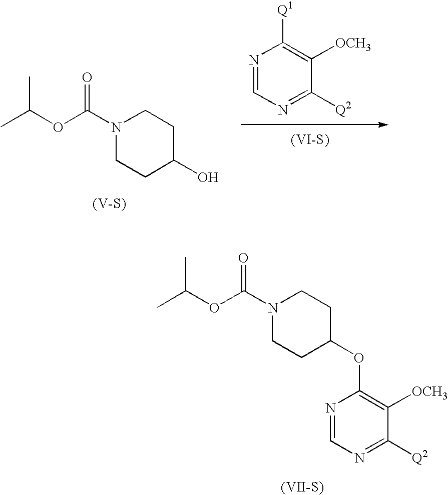
reacting a compound of formula (V-S) with a compound of formula (VI-S) wherein Q¹ and Q² are each an independently selected leaving group, in the presence of a first base, in a first organic solvent; to yield the corresponding compound of formula (VII-S); 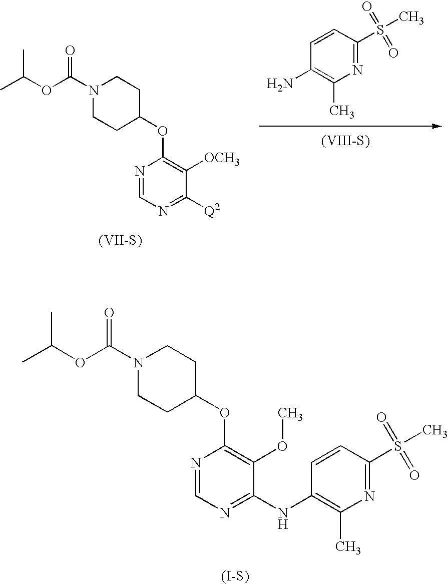
reacting the compound of formula (VII-S) with a compound of formula (VIII-S), in the presence of a second base; in the presence of a catalyst system; in a second organic solvent; to yield the corresponding compound of formula (I-S).

2. A process as in claim 1, wherein Q¹ and Q² are the same and are each chloro.

3. A process as in claim 1, wherein the compound of formula (VI-S) is present in an amount of about 1.0 molar equivalents.

4. A process as in claim 1, wherein the first base is selected from the group consisting of NaH and KO-t-Bu.

5. A process as in claim 1, wherein the first base is present in an amount in the range of from about 1.0 to about 1.2 molar equivalents.

6. A process as in claim 1, wherein the first base is KO-t-Bu and wherein the KO-t-Bu is present in an amount of about 1.1 molar equivalents.

7. A process as in claim 1, wherein the first organic solvent is toluene.

8. A process as in claim 1, wherein the second base is NaO-t-Bu.

9. A process as in claim 1, wherein the second base is present in an amount in the range of from about 1.1 to about 2.0 molar equivalents.

10. A process as in claim 1, wherein the second base is NbaO-t-Bu and wherein the NaO-t-Bu is present in an amount of about 1.5 molar equivalents.

11. A process as in claim 1, wherein the catalyst system comprises palladium and a phosphine ligand.

12. A process as in claim 11, wherein the catalyst system is a mixture of palladium acetate and bis(2-diphenylphosphinophenyl)ether.

13. A process as in claim 11, wherein the catalyst system is a mixture of 2 mol% palladium acetate and 4 mol% bis(2-diphenylphosphinophenyl)ether and wherein the catalyst system is present in an amount of about 0.02 molar equivalents.

14. A process as in claim 1, wherein the second organic solvent is toluene.

15. A process as in claim 1, wherein the compound of formula (VII-S) is reacted with the compound of formula (VIII-S) at a temperature of about 60° C.

16. A compound prepared according to a process as in claim 1.

17. A pharmaceutical composition comprising a pharmaceutically acceptable carrier and a compound prepared as in claim 16.

18. A pharmaceutical composition made by mixing a compound prepared as in claim 16 and a pharmaceutically acceptable carrier.

19. A process for making a pharmaceutical composition comprising mixing a compound prepared as in claim 16 and a pharmaceutically acceptable carrier.

20. A method of treating a metabolic related disorder comprising administering to a subject a need thereof a therapeutically effective amount of the compound prepared as in claim 16.

21. The method of claim 20, wherein the metabolic related disorder is selected from the group consisting of hyperlipidemia, type 1 diabetes, type 2 diabetes mellitus, idiopathic type 1 diabetes (Type 1b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin disorders, connective tissue disorders, foot ulcerations, ulcerative colitis, endothelial dysfunction and impaired vascular compliance.

22. The method of claim 20, wherein the metabolic related disorder is selected from the group consisting of type I diabetes, type II diabetes, inadequate glucose tolerance, insulin resistance, hyperglycemia, hyperlipidemia, hypertriglyceridemia, hypercholesterolemia, dyslipidemia and syndrome X.

23. The method of claim 20, wherein the metabolic related disorder is obesity.

24. A method of treating a metabolic related disorder selected from the group consisting of type I diabetes, type II diabetes, inadequate glucose tolerance, insulin resistance, hyperglycemia, hyperlipidemia, hypertriglyceridemia, hypercholesterolemia, dyslipidemia, syndrome X and obesity, comprising administering to a subject in need thereof a therapeutically effective amount of a compound prepared as in claim 16.

25. The use of a compound prepared as in claim 16 in a method of treatment of the human or animal body.

26. The use of a compound prepared as in claim 16 in a method of treating a metabolic related disorder.

27. The use of a compound prepared as in claim 16 in a method of treating a metabolic related disorder selected from the group consisting of Type I diabetes, Type II diabetes, inadequate glucose tolerance, insulin resistance, hyperglycemia, hyperlipidemia, hypertriglyceridemia, hypercholesterfolemia, dyslipidemia and Syndrome X.

28. The use of a compound prepared as in claim 16 for treating of Type II diabetes.

29. The use of a compound prepared as in claim 16 in a method of (a) decreasing food intake, (b) inducing satiety, (c) controlling weight gain, or (d) decreasing weight gain, in a subject in need thereof.

30. The use of a compound prepared as in claim 16 for the preparation of a medicament for treating a metabolic related disorder in a subject in need thereof.

31. The use of a compound prepared as in claim 16 for the preparation of a medicament for treating (a) type I diabetes, (b) type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, (j) syndrome X or (k), in a subject in need thereof.

32. The use of a compound prepared as in claim 16 for the preparation of a medicament for treating Type II diabetes, in a subject in need thereof.

33. The use of a compound prepared as in claim 16 for the preparation of a medicament for (a) decreasing food intake, (b) inducing satiety, (c) controlling weight gain, or (d) decreasing weight gain, in a subject in need thereof.
